(19) United States
(12) Patent Application Publication
(10) Pub. No.: US 2009/0247589 A1
(43) Pub. Date: Oct. 1, 2009

(54) Monocarboxylates for modifying macrophage function

(75) Inventors: Kambiz Zandi-Nejad, Brighton, MA (US); Anil Chandraker, Westwood, MA (US); Joseph Bonventre, Wayland, MA (US); David Mount, Newton, MA (US)

Correspondence Address: LAW OFFICE OF MICHAEL A. SANZO, LLC, 15400 CALHOUN DR., SUITE 125, ROCKVILLE, MD, 20855, US

(73) Assignee: The Brigham and Women's Hospital, Inc., Boston, MA (US)

(21) Appl. No.: 12/310,971

(22) PCT Filed: Sep. 12, 2007
(86) PCT No.: PCT/US07/19748

§ 371 Date: Mar. 13, 2009

Related U.S. Application Data
(60) Provisional application No. 60/844,374, filed on Sep. 14, 2006.

(51) Int. Cl. A61K 31/455; C12N 5/06; A61P 3/10; A61P 37/06

(52) U.S. Cl. 514/356; 435/375

(57) ABSTRACT

The present invention is directed to methods of inhibiting the production of proinflammatory agents by macrophages by contacting the cells with a monocarboxylate such as nicotinic acid. The invention also includes methods of treating diseases that are associated with macrophage activation such as atherosclerosis; systemic lupus erythematosus; rheumatoid arthritis; inflammatory bowel disease; and diabetes mellitus.

### FIELD OF THE INVENTION

The present invention is directed to methods of modifying the function of macrophages using monocarboxylates such as nicotinic acid (nicotinate), pyruvic acid (pyruvate), lactic acid (lactate), propionic acid (propionate), β-hydroxybutyric acid (β-hydroxybutyrate) and acifran or its derivatives. These agents have been found to inhibit the production of proinflammatory agents by macrophages and should therefore be useful in the management of a wide variety of diseases and conditions.

### BACKGROUND OF THE INVENTION

Chronic kidney disease (CKD) affects more than 20 million people in the United States and many more people worldwide (Levey, et al. Ann. Intern. Med. 139 (2):137-147 (2003)). The final common pathway for all chronic nephropathies is renal fibrosis, with macrophage infiltration being a constant feature (both clinically and experimentally) and central to the process (Wilson, et al., Curr. Opin. Nephrol. Hypertens. 13 (3): 285-90 (2004)). Macrophage depletion is associated with significantly reduced renal injury and macrophage repletion can restore renal injury in a dose-dependent manner. In the kidney, macrophages are the main source of the proinflammatory cytokine interleukin-1β (IL-1β), a stimulant for epithelial mesenchymal transformation (EMT) of proximal tubule cells (Vesey, et al., Kidney Int. 62 (1):31-40 (2002)). These factors suggest that methods of modifying macrophage function may be useful in the treatment of chronic kidney disease.

In addition to contributing to chronic kidney disease, the production of proinflammatory agents by monocytes and macrophages has been associated with a wide variety of other diseases, including but not limited to: atherosclerosis; systemic lupus erythematosus; rheumatoid arthritis; inflammatory bowel disease; acute and chronic solid organ transplant rejection; and diabetes mellitus (both type 1 and type 2). Agents that are capable of modifying macrophage function should be useful in the treatment of all these conditions.

Niacin (nicotinic acid), a water-soluble vitamin readily filtered by the kidney, in pharmacological doses, is a lipid-lowering drug that uniquely improves all lipoprotein abnormalities, including increasing HDL cholesterol (McKenney, Arch. Intern. Med. 164 (7):697-705 (2004)). Although niacin's role in the treatment of hyperlipidemia has recently been overshadowed by the emergence of newer medications, particularly statins, recent data supports the combined use of niacin and statins for an improved outcome (Taylor, et al., Circulation 110 (23):3512-3517 (2004)).

### SUMMARY OF THE INVENTION

The present invention is based upon the discovery that monocarboxylate compounds (MCs) (e.g., nicotinate) can modulate macrophage function and mitigate proinflammatory stimulation of these cells, e.g., by uric acid and interferon-gamma (IFN-γ). There have also been indications that nicotinate infusion may have a beneficial effect in a chronic rejection model of murine cardiac transplant.

In its first aspect, the invention is directed to a method of modifying the function of macrophages and their production of proinflammatory agents by treating these cells with an effective amount of a monocarboxylate. For the purposes of the present invention, unless otherwise indicated, the term “macrophages” includes macrophages, monocytes, and dendritic cells. The term “proinflammatory agent” refers to any of the various agents known to promote inflammation that are produced and/or released by macrophages, including, but not limited to, interleukin-6 (IL-6), interleukin-1β (IL-1β) and tumor necrosis factor alpha (INF-α). The term “monocarboxylate” refers to small compounds having a single carboxyl group, with the most preferred compounds being nicotinic acid, β-hydroxybutyrate, propionate, pyruvate, lactate and acifran. An “effective amount” is a sufficient amount of compound to significantly alter macrophage function and reduce the amount of proinflammatory agent produced/released by macrophages when stimulated by an activating agent such as uric acid, interferon gamma or lipopolysaccharide (LPS). The method may be used in vivo or in vitro by scientists studying macrophage function.

In another aspect, the invention is directed to a method of treating a patient for a disease or condition associated with macrophage activation and/or the production/secretion of proinflammatory agents from macrophages (excluding foam cell macrophages seen in atherosclerosis associated with an abnormal lipid profile), by administering a therapeutically effective amount of a monocarboxylate. A “therapeutically effective amount” is a sufficient amount of drug to cause an improvement in at least one symptom/sign associated with a particular disease or condition or reduction in an inflammatory marker such as C-Reactive Protein (CRP). Alternatively, the term may refer to a sufficient amount of drug to reduce the likelihood of a disease or condition arising. For example in the case of niacin, it is expected that between 20 and 100 μmol/kg/day will be given to patients, and more preferably, between 40 and 400 μmol/kg/day; for other monocarboxylates the dose may be different depending on the characteristics of the molecule. It is expected that niacin will be administered in an oral unit dosage form e.g., a tablet or capsule, with between about 100 and 1000 mg of monocarboxylate present. The term “unit dosage form” refers to a single drug administration entity such as a single tablet.

Any of the various diseases known to be caused or exacerbated by macrophage activation and/or the production/secretion of proinflammatory agents from macrophages may be treated using the methods described above. This includes but is not limited to autoimmune diseases, solid organ transplantation, progression of chronic kidney disease, cardiovascular diseases and inflammatory diseases. More specifically, it includes chronic kidney disease; atherosclerosis occurring in patients in the absence of abnormal blood levels of lipids or lipoproteins; systemic lupus erythematosus; rheumatoid arthritis; diabetes mellitus (either type 1 or type 2); and inflammatory bowel disease. In addition, the method may be used to treat ischemia/reperfusion injury, sepsis-associated acute kidney injury (formerly known as acute renal failure) and to prevent either chronic or acute organ transplant rejection in patients. In all cases, the most preferred monocarboxylates are nicotinic acid, β-hydroxybutyrate, propionate, pyruvate, lactate and acifran.

Due to a loss of monocarboxylates during dialysis, patients with temporary or permanent renal failure will experience an increase in proinflammatory activity that, inter alia, increases the risk of atherosclerotic plaques forming, progressing or rupturing. Thus, dialysis patients, especially those with atherosclerosis or other cardiovascular diseases, are an especially preferred group for monocarboxylate treatment. Patients should typically be administered 500-3000 mg of monocarboxlate, such as nicotinic acid or acifran, preferably in an extended release dosage form, i.e., in a dosage form where drug is continuously released over a period of several hours or days.

It will be understood that, in all of the methods described herein, any pharmaceutically acceptable form of monocarboxylate may be used, including the acid form or any pharmaceutically acceptable salt. Unless otherwise indicated, reference to the acid or salt will be understood to also include all of the other pharmaceutically acceptable forms of the drug as well. Those of skill in the art will also recognize derivatives and analogues of these compounds that may be used in their place and which would be considered equivalents. Among the most preferred of the monocarboxylates are nicotinic acid and β-hydroxybutyrate given at a dose of 40-400 μmol/kg/day or a functionally equivalent dose, respectively.

In another aspect, the invention is directed to a therapeutic composition or kit having both a monocarboxylate drug, e.g., any of the various drugs mentioned above, and instructions for administering this drug to a patient to treat a disease associated with macrophage activation and/or the production of proinflammatory agents by macrophages. The monocarboxylate drug should be part of a pharmaceutical composition in unit dosage form and be packaged in a finished pharmaceutical container. The term “finished pharmaceutical container” refers to any of the various types of packaging typically used for pharmaceuticals, such as bottles, vials, blister packs, etc. Each unit dosage form will typically have between 100 and 1,000 mg of nicotinic acid (or the equivalently effective dose of another monocarboxylate), and more typically between 150 and 500 mg. Especially preferred are slow release tablets or capsules having 500-1000 mg of drug, especially nicotinic acid or acifran.

The instructions that form a part of the therapeutic compositions may appear on packaging containing the monocarboxylate drug, on the finished pharmaceutical container or as a separate package insert. The instructions will include the dosage of monocarboxylate that should be administered to a patient to treat or prevent one or more of the various diseases or conditions described above.

### DETAILED DESCRIPTION OF THE INVENTION

The present invention is based upon experiments suggesting that monocarboxylates, especially nicotinic acid, inhibit the LPS or interferon gamma plus uric acid-induced production of proinflammatory agents from RAW 264.7 cells, a murine macrophage-like cell line. This has led to the basic concept that these compounds should be capable of providing a therapeutic benefit in diseases and conditions in which macrophages play a contributory role. One advantage of treatments using monocarboxylates is that some of these compounds, e.g., niacin (nicotinic acid), have been marketed for many years and are known to be safe for ingestion.

A. Monocarboxylates

Monocarboxylates are a group of relatively small organic molecules with one carboxyl group. They typically have a molecular weight of less than 500 and fewer than 25 carbons. Structurally, they will generally have the formula R₁—(R₂)ₙ-CO₂H, where n is 0 or 1; and R₁ and R₂ are each independently: a C₁-C₆ straight or branched alkyl or alkoxy; an aryl; a heteroaryl containing one or more heteroatoms selected from O, N and S; a C₃-C₆ carbocyclic ring, a C₃-C₆ heterocyclic ring containing one or more heteroatoms selected from O, N and S; and wherein said alkyl, alkoxy, aryl, heteroaryl, carbocyclic ring or heterocyclic ring may each independently be substituted by one or more substituents selected from OH, a halogen, NO₂, and CF₃. Nicotinic acid (nicotinate), propionic acid (propionate), lactic acid (lactate), β-hydroxybutyric acid (β-hydroxybutyrate) and acifran are all monocarboxylates and are the preferred compounds for use in the methods described herein. These compounds have all been known in the art for many years and may be purified or synthesized using any of a variety of methods that have been described. They are all available commercially and nicotinic acid (also known as niacin or vitamin B3) has been reported to be of value in the treatment of hyperlipidemia. Some representative structures are as follows:

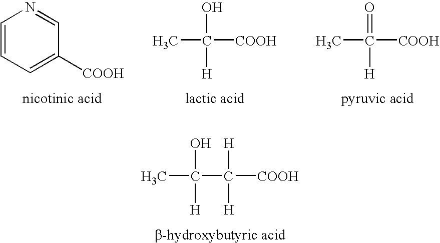

B. Making of Pharmaceutical Compositions

Monocarboxylate compounds may be incorporated into pharmaceutical compositions in accordance with methods that are standard in the art (see e.g., Remington's Pharmaceutical Sciences, Mack Publishing Co., (1990)). Formulations may be designed for delivery by any of the routes commonly used in the art, with preparations designed for oral delivery being preferred. For oral compositions, e.g. tablets or capsules, the monocarboxylate drug should typically be present in an amount functionally equivalent to 100 to 1000 mg of niacin. Although not preferred, other routes of administration may also be employed.

Monocarboxylates may be used in conjunction with any of the vehicles and excipients commonly employed in pharmaceutical preparations including water, salt solutions, alcohols, gum arabic, vegetable oils, benzoalcohols, polyethylene glycol, gelatin, carbohydrates such as lactose, amylase, or starchy magnesium stearate; talc; salycic acid; paraffin; fatty acid esters; polymers; etc. The pharmaceutical preparations can be sterilized and, if desired, mixed with auxiliary agents such as: dispersants; lubricants; preservatives; stabilizers; wetting agents; emulsifiers; salts for influencing osmotic pressure; buffers; coloring agents; flavoring agents; and/or aromatic substances.

Solutions, particularly solutions for injection, can be prepared using water or physiologically compatible organic solvents such ethanol; 1,2-propylene glycol; polygycols; dimethylsulfoxides; fatty alcohols; triglycerides; partial esters of glycerine; and the like. The preparations can be made using conventional techniques that may include sterile isotonic saline, water, 1,3-butanediol, ethanol, 1,2-propylene glycol, polygycols mixed with water, Ringer's solution, etc.

C. Dosage Forms and Routes of Administration

The present invention is compatible with any route of administration including oral, internal, rectal, nasal, sublingual, transdermal, vaginal, intravenous, intraarterial, intramuscular, intraperitoneal, via inhalation, and subcutaneous routes. Dosage forms that may be used include tablets, capsules, powders, aerosols, suppositories, skin patches, parenterals, sustained release preparations and oral liquids, including suspensions solutions and emulsions. If desired, compositions, particularly compositions for injection, may be freeze-dried and lyophilizates reconstituted before administration. Dosage forms may include monocarboxylates as the sole active ingredient or they may include other active agents as well. All dosage forms may be prepared using methods that are standard in the art and that are taught in reference works such as Remington's Pharmaceutical Sciences (Oslo, A. ed., Mack Publishing Co. (1990)).

D. Treatment Methods

The therapeutic objective of the methods described herein is to manage, treat, slow the progression, or prevent the development of one of the diseases or conditions described herein. In the case of an existing disease, successful treatment will be reflected in an improvement in one or more of the symptoms/signs associated with the disease. For example, in the treatment of an inflammatory condition, sufficient drug should be provided to reduce pain or swelling associated with inflammation. In CKD, sufficient drug should be administered to improve GFR, slow the rate of its decline, or reduce the filtration of substances that should normally be retained, e.g., albumin. In cardiovascular disease and malnutrition inflammation and atherosclerosis (MIA) syndrome, treatment should reduce the level of inflammatory biomarkers, e.g. CRP, in patients, reduce mortality and/or reduce the frequency of cardiovascular events.

When used to prevent the development of a disease, the dose administered will be based upon the results of animal studies and clinical studies performed using methods well known in the art. Monocarboxylate drugs are already available for the treatment of other conditions, e.g., hyperlipidemia, and existing dosages may serve as a starting point for evaluating dosages effective in preventing other macrophage-associated diseases. Based upon existing knowledge, it is expected that, using oral delivery methods, a patient will typically receive an oral dose of a monocarboxylate equivalent to 500 to 3000 mg (and more preferably 500-2000) of nicotinic acid per day. Extended release forms of drugs are especially preferred.

The patient population selected for administration of pharmaceutical compositions containing the monocarboxylates will be people that have, or who are known to be at high risk for developing, a macrophage-associated disease or condition. In all cases, treatment methods and dosages will be selected by the attending physician based upon clinical considerations using methods that are well-known in the art.

E. Kidney Dialysis Patients

The most severe form of chronic kidney disease or end stage renal disease (ESRD) requires some form of renal replacement therapy (dialysis or kidney transplant) for survival. Age and gender adjusted cardiovascular disease (CVD) mortality is 10-30 fold higher in dialysis patients compared with the general population (Foley, et al., J. Am. Soc. Nephrol. 9:S16-23 (1998)). Some of the risk factors for CVD in dialysis patients are different from those of the general population and, in some cases, even appear to be reversed (Foley, et al., J. Am. Soc. Nephrol. 9:S 16-23 (1998); Lowrie, et al., Am. J. Kidney Dis. 15:458-82 (1990); Colhoun, et al., N. Engl. J. Med. 353:238-48 (2005)). This lack of a positive association between some traditional risk factors (e.g., cholesterol level) and mortality can be explained, in part, by the systemic inflammation commonly seen in dialysis patients (Liu, et al., JAMA 291:451-9 (2004); Stenvinkel, et al., Nephrol. Dial. Transplant 15:953-60 (2000); Am. J. Kidney Dis. 45:S1-153 (2005); Stenvinkel, et al. Kidney Int. 55:1899-911 (1999); Zoccali, et al., J. Hypertens. 18:1207-13 (2000)).

Macrophages and their proinflammatory products (e.g., cytokines) play a central role in the pathogenesis of atherosclerotic CVD and the chronic inflammatory state commonly seen in dialysis patients. On the other hand, macrophages are essential to the process of repair. This paradox can be explained in part by their ability to produce both pro- and anti-inflammatory cytokines the dominance of which depends on the balance between pro- and anti-inflammatory signals macrophages receive. Our studies suggest that nicotinic acid and other monocarboxylates can modify macrophage function and reduce their proinflammatory cytokine production, probably through PUMA-G, a novel Gᵢ-protein coupled receptor primarily expressed in adipocytes and macrophages. All the known ligands of PUMA-G are monocarboxylates (e.g., nicotinic acid), which are water soluble, readily dialyzable molecules (Tunaru, et al., Nat. Med. 9:352-355 (2003); Taggart, et al. J. Biol. Chem. 280:26649-26652 (2005)). Thus, endogenous monocarboxylate depletion by dialysis will tip the balance in favor of a proinflammatory state in macrophages and thereby promote atherosclerotic CVD in these patients. Replacement by nicotinic acid can be used to restore the balance and hence reduce the cardiovascular risks associated with dialysis. In addition to dialysis patients where this phenomenon is most pronounced, treatment with nicotinic acid will be of considerable importance in the general population, where 20% of cardiovascular events occur in patients with no traditional risk factors and 50% occur in patients with a normal lipid profile.

F. Packaging of Therapeutic Compositions

Pharmaceutical compositions containing monocarboxylate drugs may be placed in a finished pharmaceutical container and sold along with instructions to physicians regarding the use of the compositions in treating or preventing any of the diseases described herein. Depending upon the intended route of delivery, containers may include bottles, vials, ampoules, blister packs etc.

Instructions concerning the use of pharmaceutical compositions may be included on the container with the pharmaceutical composition or as a package insert. Alternatively, the instructions may be included on a box or other package in which the pharmaceutical composition is sold. In all cases, the instructions will indicate that the pharmaceutical compositions are to be administered for the purpose of preventing or treating one or more macrophage-associated diseases or conditions including but not limited to: atherosclerosis; chronic kidney disease, systemic lupus erythematosus; rheumatoid arthritis; inflammatory bowel disease; diabetes mellitus (both type 1 and type 2); and acute and chronic rejection of solid organ transplant. A description of the active ingredient(s) will also be included along with information concerning dosage and how the pharmaceutical composition should be administered.

### EXAMPLES

### Effects of Monocarboxylates and Uric Acid on Macrophage Function

Our data shows that in RAW 264.7 cells, a murine macrophage-like cell line, uric acid collaborates with other macrophage stimulators such as IFN-γ synergistically, further stimulating production of proinflammatory cytokines such as TNF-α. This effect is largely abrogated by concomitant administration of nicotinic acid or other monocarboxylates. The production of proinflammatory cytokines, e.g., TNF-α and IL-6, by LPS-stimulated RAW 264.7 cells is also significantly reduced by the concomitant administration of nicotinic acid. It is of note, that addition of neither uric acid nor any of the monocarboxylates, was associated with a change in pH measured up to 48 hours post administration.

Results have also suggested that neither IFN-γ+ uric acid nor nicotinic acid have any significant effect on cell proliferation in RAW 264.7 cells measured by MTT assay. In addition, in a chronic rejection model of murine cardiac transplantation treated with either nicotinic acid or saline, as continuous infusion by osmotic minipumps for 4 weeks, nicotinate treated animals (except for those which excluded as outlyers probably due to technical problem associated with surgery) had near normal grafts compared to the saline-treated controls, which had evidence of myocyte loss and moderate mononuclear cell infiltrate in the interstitium and perivascular areas. The cellular infiltrate was strongly CD68+, indicating heavy macrophage infiltration. The dose of nicotinic acid was 60-80 μmol/kg/day, corresponding to 500-1000 mg of nicotinic acid in a 70 kg human (depending on bioavailability).

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

1-25. (canceled)

26. A method of inhibiting the production of a proinflammatory agent by macrophages, comprising treating said macrophages with an effective amount of a monocarboxylate.

27. The method of claim 26, wherein said proinflammatory agent is selected from the group consisting of interleukin-6; interleukin-1β; and tumor necrosis factor alpha (TNF-α).

28. The method of claim 26, wherein said monocarboxylate is selected from the group consisting of: nicotinic acid; β-hydroxybutyrate; pyruvate; propionate; lactate and acifran.

29. The method of claim 28, wherein said monocarboxylate is either nicotinic acid or β-hydroxybutyrate.

30. A method of treating a patient for a disease or condition associated with the production of a proinflammatory agent by macrophages, comprising administering a therapeutically effective amount of a monocarboxylate drug to said patient.

31. The method of claim 30, wherein said monocarboxylate is selected from the group consisting of: nicotinic acid; β-hydroxybutyrate; propionate; pyruvate; lactate; and acifran.

32. The method of claim 31, wherein said monocarboxylate is administered either orally or parenterally at a dose equivalent to 20-100 μmol/kg/day of nicotinic acid.

33. The method of claim 31, wherein said monocarboxylate is administered at a dose of 40-400 μmol/kg/day.

34. The method of claim 31, wherein said monocarboxylate is administered to said patient orally in unit dose form, each unit dose comprising 100-1000 mg of monocarboxylate.

35. The method of claim 30, wherein said disease or condition is an autoimmune disease, cardiovascular disease, inflammatory disease, or chronic kidney disease.

36. The method of claim 30, wherein said disease or condition is atherosclerosis unassociated with abnormal blood levels of lipids or lipoproteins.

37. The method of claim 30, wherein said disease or condition is systemic lupus erythematosus, rheumatoid arthritis, or inflammatory bowel disease.

38. The method of claim 30, wherein said disease or condition is diabetes mellitus type 1 or type 2.

39. The method of claim 30, wherein said disease or condition is ischemia/reperfusion injury or sepsis-associated acute kidney injury.

40. The method of claim 30, wherein said patient is an organ transplant patient and said monocarboxylate is administered to prevent organ rejection (acute or chronic).

41. A method of treating a dialysis patient to reduce the risks associated with increased proinflammatory macrophage activity comprising administering to said patient a therapeutically effective amount of a monocarboxylate drug.

42. The method of claim 41, wherein said monocarboxylate is selected from the group consisting of: nicotinic acid; β-hydroxybutyrate; propionate; pyruvate; lactate; and acifran.

43. The method of claim 42, wherein said monocarboxylate is administered either orally or parenterally at a dose equivalent to 20-100 μmol/kg/day of nicotinic acid.

44. The method of claim 42, wherein said monocarboxylate is administered at a dose of 40-400 μmol/kg/day.

45. The method of claim 42, wherein said monocarboxylate is administered to said patient orally in unit dose form, each unit dose comprising 100-1000 mg of monocarboxylate.
